(19) United States
(12) Patent Application Publication
(10) Pub. No.: US 2024/0429609 A1
(43) Pub. Date: Dec. 26, 2024

(54) COLLAPSIBLE COIL ANTENNA

(71) Applicant: EDWARDS LIFESCIENCES CORPORATION, Irvine, CA (US)

(72) Inventor: Yaron Keidar, Kiryat Ono (IL)

(21) Appl. No.: 18/828,165

(22) Filed: Sep. 9, 2024

Related U.S. Application Data
(63) Continuation of application No. PCT/US2023/014650, filed on Mar. 6, 2023.
(60) Provisional application No. 63/319,249, filed on Mar. 11, 2022.

(51) Int. Cl. H01Q 7/02; A61F 2/24; A61N 1/362; A61N 1/372; A61N 1/378; H01Q 1/27

(52) U.S. Cl. CPC H01Q 7/02; A61F 2/2418; A61N 1/362; A61N 1/37229; A61N 1/3787; H01Q 1/273

(57) ABSTRACT

Collapsible coil antennas for implantable medical devices are described. Collapsible coil antennas are capable of expanding and contracting without breaking, while maintaining their functionality as antennas. In many aspects, they are made from a drawn filled tube having three layers, and the drawn filled tube is formed into the shape of a crown.

### CROSS-REFERENCE TO RELATED APPLICATIONS

The current application claims the benefit of and priority to PCT Application No. PCT/US2023/014650, entitled “COLLAPSIBLE COIL ANTENNA,” filed Mar. 6, 2023, which claims the benefit of and priority to U.S. Provisional Patent Application No. 63/319,249 entitled “COLLAPSIBLE COIL ANTENNA” filed Mar. 11, 2022. The disclosures of PCT Application No. PCT/US2023/014650 and U.S. Provisional Patent Application No. 63/319,249 are hereby incorporated by reference in their entirety for all purposes.

### FIELD OF THE INVENTION

This invention generally relates implantable antennas for communication and wireless power transfer.

### BACKGROUND

Radio frequency communication involves the transmission and reception of radio frequency electromagnetic signals. Antennas are used by transmitters and receivers to send and receive the signal.

Antennas can also be used for transmitting and receiving electromagnetic energy for the purposes of wireless power transfer.

### SUMMARY OF THE INVENTION

Collapsible coil antennas in accordance with aspects of the invention are illustrated. One aspect includes a collapsible coil antenna for implantation into a body, including a drawn filled tube having three layers: an inner most layer being a conductive layer, a middle layer being a jacket layer, and an outer layer being an insulation layer.

In another aspect, the conductive layer is made of a material selected from the group consisting of: copper, gold, and silver.

In a further aspect, the jacket layer is made of a material selected from the group consisting of: stainless steel, nitinol, and cobalt-chrome.

In still another aspect, the insulation layer is made of a material selected from the group consisting of: polyurethane and parylene C.

In a still further aspect, the collapsible coil antenna is capable of expanding from a collapsed state.

In yet another aspect, the antenna is capable of being used to wirelessly harvest power.

In a yet further aspect, the antenna is capable of being used to wirelessly communicate.

In another additional aspect, the collapsible coil antenna is part of an assembly further comprising a stent.

In a further additional aspect, the collapsible coil antenna is part of an assembly further comprising an artificial heart valve.

In another aspect again, the collapsible coil antenna is configured to collapse during an implantation procedure during delivery to a target implantation site, and expanded at the target implantation site.

In a further aspect again, the drawn filled tube is formed into a crown shape.

In still yet another aspect, the collapsible coil antenna is connected to at least one implantable medical device.

In a still yet further aspect, the at least one implantable medical device is a pacemaker.

In still another additional aspect, the at least one implantable medical device is a sensor.

In a still further additional aspect, the drawn filled tube has a diameter of 0.1 mm to 35 mm.

In still another aspect again, the drawn filled tube has a diameter of 7 mm and a height of 5 mm.

In a still further aspect again, a wirelessly powered pacemaker, including a pacemaker, and a collapsible coil antenna comprising a drawn filled tube having three layers, where the three layers are: an inner most layer being a conductive layer, a middle layer being a jacket layer, and an outer layer being an insulation layer, and where the collapsible coil antenna wirelessly harvests power and provides the harvested power to the pacemaker.

In yet another additional aspect, the pacemaker is configured to receive control instructions via a modulated signal received by the collapsible coil antenna.

In a yet further additional aspect, a wirelessly powered implantable sensor, including a sensor, and a collapsible coil antenna comprising a drawn filled tube having three layers, where the three layers are: an inner most layer being a conductive layer, a middle layer being a jacket layer, and an outer layer being an insulation layer, and where the collapsible coil antenna wirelessly harvests power and provides the harvested power to the sensor.

In yet another aspect again, the sensor provides recorded data to an external device via the collapsible coil antenna.

Additional aspects and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

FIG. 1A illustrates a collapsible coil antenna in an expanded state in accordance with an aspect of the invention.

FIG. 1B illustrates the collapsible coil antenna in a collapsed state in accordance with an aspect of the invention.

FIG. 2 illustrates a collapsible coil antenna wrapped around a stent in accordance with an aspect of the invention.

### DETAILED DESCRIPTION

Implantable medical devices are often life saving and life maintaining, but also tend to require electrical power. Given the implanted nature of these devices, providing the power is not an easy task. Recently, wireless power transfer has been used to power implantable medical devices which are equipped with coil antennas capable of harvesting electromagnetic energy to power the attached device. A varying electromagnetic field from a power generator induces a current to the antenna which then is used to power an attached medical device. However, the amount of power which can be transferred is correlated with the area inside the coil of the antenna. In small implants, the size of the antenna tends to be small as well.

Some implantable medical devices like vascular stents and transcatheter heart valves are designed to be collapsible. That is, they are designed to be delivered into the body in a collapsed, small diameter state. Once inside the body, they can expand to a larger diameter state. This is helpful for implantation, but poses physical problems for conventional coil antennas. While a collapsible antenna would have a larger area when deployed to a larger diameter inside the body, the expansion process is likely to break conventional coil antennas. Antennas are typically made of good electrical conductors like copper, gold, and silver. These metals tend to snap under the forces involved in crimping and expanding implants.

Collapsible coil antennas described herein are capable of expanding without significant risk of breakage. In many aspects, collapsible coil antennas are constructed out of a drawn filled tube having three layers: an inner conductive core, a middle metal jacket, and an outer polymer insulation layer. In numerous aspects, the conductive core is made of copper, gold, silver, or another conductive metal. The metal jacket can be constructed from a stainless steel, nitinol, cobalt-chrome, or another protective metal. The polymer insulation layer can be made of polyurethane, parylene C, or another body-safe insulator. Any combination of the above materials can be used without departing from the scope or spirit of the invention.

In numerous aspects, the drawn filled tube has a diameter of 0.5 mm or less. In various aspects, the collapsible coil antenna has a diameter of 7 mm with a 5 mm height. However, the diameter can be increased up to 35 mm for certain applications, e.g. for applications in valves. Similarly, height can be modified depending on the size of the vessel in which the antenna is to be implanted. In numerous aspects, the collapsible coil antenna is sufficiently large to fit around a stent. In numerous aspects, the collapsible coil antenna has a crown shape similar to that of a crown stent.

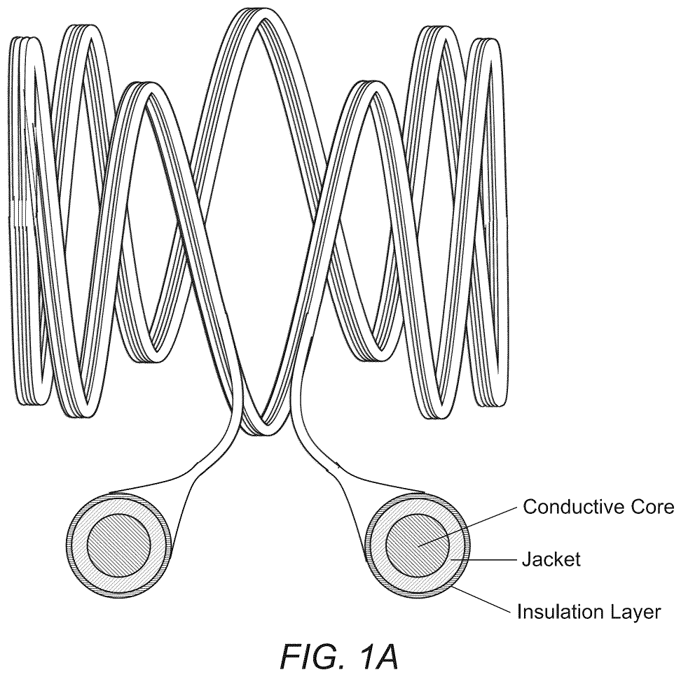

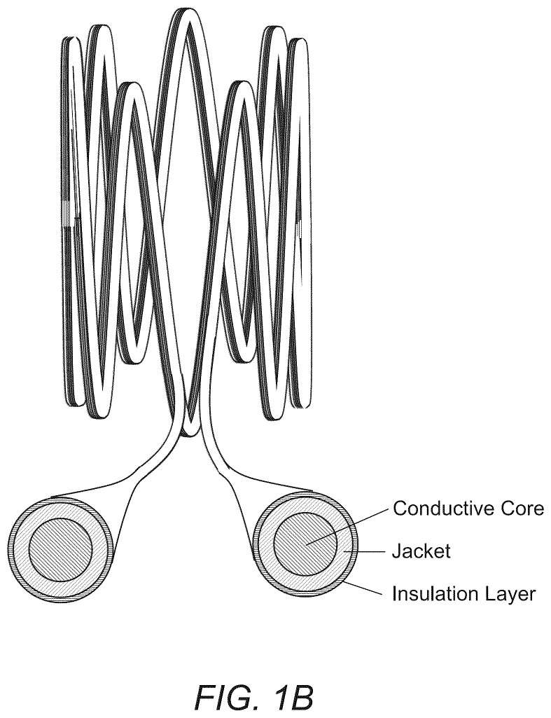

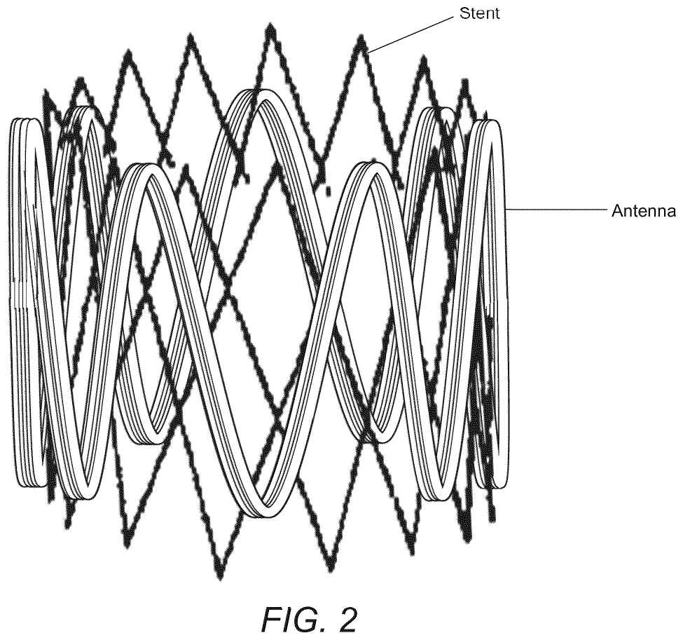

Turning now to FIG. 1A, a collapsible coil antenna having a crown structure in its expanded state in accordance with an aspect of the invention is illustrated. The drawn filled tube is cross-sectioned for understanding, but it is to be further understood that the cross-sectional view is not a protruding portion of the antenna. FIG. 1B shows the collapsible coil antenna in its contracted state in accordance with an aspect of the invention. Further, FIG. 2 illustrates a collapsible coil antenna wrapped around a stent which may also be expanded or contracted as desired.

The power harvested using collapsible coil antennas can be used to power any number of different implantable medical devices including (but not limited to) pacemakers, sensors, neurostimulators, and/or any other medical device as appropriate to the requirements of specific applications of aspects of the invention. Further, multiple collapsible coil antennas can be coupled with an implanted device in order to provide additional power. Further, collapsible coil antennas can be used to transmit data between external devices and implantable devices by modulating the power transfer signal using a code (e.g. pulse-width modulation, phase shift keying, quadrature amplitude modulation, etc.) that is understandable by the receiving device. Similarly, in various aspects, the collapsible coil antenna can be used by an implantable device to transmit data to external devices. In this way, the collapsible coil antenna can be used to power implantable devices, control implantable devices, and/or receive data from implantable devices.

While specific materials and dimensions are listed above, as can readily be appreciated, the dimensions may be modified as needed for specific implants, and the materials may be exchanged for materials with similar properties without departing from the scope or spirit of the invention. Thus, aspects of the present invention should be considered in all respects as illustrative and not restrictive. Accordingly, the scope of the invention should be determined not by the aspects illustrated, but by the appended claims and their equivalents.

What is claimed is:

1. A collapsible coil antenna for implantation into a body, comprising:
a drawn filled tube having three layers:
an inner most layer being a conductive layer;
a middle layer being a jacket layer; and
an outer layer being an insulation layer.

2. The collapsible coil antenna of claim 1, wherein the conductive layer is made of a material selected from the group consisting of: copper, gold, and silver.

3. The collapsible coil antenna of claim 1, wherein the jacket layer is made of a material selected from the group consisting of: stainless steel, nitinol, and cobalt-chrome.

4. The collapsible coil antenna of claim 1, wherein the insulation layer is made of a material selected from the group consisting of: polyurethane, and parylene C.

5. The collapsible coil antenna of claim 1, wherein the collapsible coil antenna is capable of expanding from a collapsed state.

6. The collapsible coil antenna of claim 1, wherein the antenna is capable of being used to wirelessly harvest power.

7. The collapsible coil antenna of claim 1, wherein the antenna is capable of being used to wirelessly communicate.

8. The collapsible coil antenna of claim 1, wherein the collapsible coil antenna is part of an assembly further comprising a stent.

9. The collapsible coil antenna of claim 1, wherein the collapsible coil antenna is part of an assembly further comprising an artificial heart valve.

10. The collapsible coil antenna of claim 1, wherein the collapsible coil antenna is configured to collapse during an implantation procedure during delivery to a target implantation site, and expanded at the target implantation site.

11. The collapsible coil antenna of claim 1, wherein the drawn filled tube is formed into a crown shape.

12. The collapsible coil antenna of claim 1, wherein the collapsible coil antenna is connected to at least one implantable medical device.

13. The collapsible coil antenna of claim 12, wherein the at least one implantable medical device is a pacemaker.

14. The collapsible coil antenna of claim 12, wherein the at least one implantable medical device is a sensor.

15. The collapsible coil antenna of claim 1, wherein the drawn filled tube has a diameter of 0.1 mm to 35 mm.

16. The collapsible coil antenna of claim 1, wherein the drawn filled tube has a diameter of 7 mm and a height of 5 mm.

17. A wirelessly powered pacemaker, comprising:
a pacemaker, and
a collapsible coil antenna comprising a drawn filled tube having three layers, where the three layers are:
an inner most layer being a conductive layer;
an middle layer being a jacket layer; and
an outer layer being an insulation layer; and
where the collapsible coil antenna wirelessly harvests power and provides the harvested power to the pacemaker.

18. The wirelessly powered pacemaker of claim 17, wherein the pacemaker is configured to receive control instructions via a modulated signal received by the collapsible coil antenna.

19. A wirelessly powered implantable sensor, comprising:
a sensor, and
a collapsible coil antenna comprising a drawn filled tube having three layers, where the three layers are:
an inner most layer being a conductive layer;
an middle layer being a jacket layer; and
an outer layer being an insulation layer; and
where the collapsible coil antenna wirelessly harvests power and provides the harvested power to the sensor.

20. The wirelessly powered pacemaker of claim 17, wherein the sensor provides recorded data to an external device via the collapsible coil antenna.
